(19) United States
(12) Patent Application Publication
(10) Pub. No.: US 2003/0129677 A1
(43) Pub. Date: Jul. 10, 2003

(54) Diagnostic method for screening complement regulatory protein levels

(76) Inventors: Mark G. Martens, Jenks, OK (US); Anil K. Kaul, Plymouth, MN (US); Rashmi Kaul, Plymouth, MN (US)

Correspondence Address: SCHWEGMAN, LUNDBERG, WOESSNER & KLUTH, P.A., P.O. BOX 2938, MINNEAPOLIS, MN, 55402, US

(21) Appl. No.: 10/292,130

(22) Filed: Nov. 12, 2002

Related U.S. Application Data
(63) Continuation of application No. PCT/US01/14769, filed on May 9, 2001.
(60) Provisional application No. 60/203,967, filed on May 12, 2000.

(51) Int. Cl. G01N 33/574; G01N 33/53

(52) U.S. Cl. 435/7.23; 435/7.5

(57) ABSTRACT

The invention provides for a method for the early diagnosis of a premalignant lesion, prognosis of a malignant lesion and a kit for use in more rapid identification of predisposition for malignancy.

### CROSS-REFERENCE TO RELATED APPLICATIONS

This application is a continuation of PCT/US01/14769, filed on May 9, 2001, which claimed priority under 35 U.S.C. 119(e) of U.S. Provisional Application Serial No. 60/203,967, filed May 12, 2000, which applications are incorporated herein by reference.

### BACKGROUND OF THE INVENTION

In the United States, a women has one-in-nine chance of developing breast cancer during her lifetime. In 1993, one of every three new cancers in women (32%) was of the breast. It is the most common cancer in women (183,000 cases in 1993) and their second leading cause of death from cancer (46,000 in 1993) (Berg et al., 1995, Breast Cancer. Cancer (suppl, 75(1):257-269). Breast cancer is a disease whose frequency as well as pathological characteristics vary markedly with age and sex. Women develop breast cancer with an incidence of about 100 times the frequency in men. In women, the incidence of breast cancer increases with age, but rate of increase drops off sharply at the age of menopause (Pike et al., 1981, Banbury Report 8: Hormones and Cancer, Cold Spring Harbor Lab, pgs. 3-21).

Breast cancer can only be life threatening when it become invasive, at which point it carries potential for spreading and metastasis. It is critical to distinguish invasive carcinomas from noninvasive lesions, including ductal carcinoma in situ (DCIS), which represents an early, pre-invasive stage in the development of invasive breast carcinoma. While this distinction is usually made based on histologic evaluation alone, in a small but significant number of cases, accurate diagnosis may be impossible, particularly in the context of core needle biopsies. Yaziji H, et al. Adv Anat Pathol March 2000; 7(2):100-9. A standardized pathologic staging and grading system, however, does not exist for breast cancer. Uncertainties in the prognosis for patients with DCIS have caused a controversial discussion about adequate treatment, and it is suspected that most patients undergoing mastectomy may be over-treated. In order to improve treatment and treatment decision, it would be highly desirable to identify prognostic markers and therapeutic targets for DCIS. Schulze-Garg C, et al. Oncogene Feb. 21, 2000; 19(8):1028-37.

Endometrial cancer is the most common invasive neoplasm of the female genital tract and is ranked fourth in age-adjusted cancer incidence among women in the United States (Parker, S. L., et al., Cancer statistics, 47:5-27 (1997)). In 1997, uterine corpus cancer accounted for about 5.8% of female cancer incidence (an estimated 35,000 cases) and 2.2% of female cancer mortality (an estimated 6000 deaths) in the United States (Mass, H., Epidemiologiebgynakologischer Tumoren, in Wulff, K. H., Schmidt-Mattiesen, H. (eds.), Klinik der Frauenheilkunde und Geburtshilfe, Munchen, Urban & Schwarzenberg, 10:23-40 (1985)). Although the death rates for the disease have declined by more than 60% since the 1950s, incidence has shown a marked increase during the 1970s secondary to the use of high dose unopposed estrogens during that period.

Endometrial carcinoma is usually diagnosed in its early stages because most women quickly report postmenopausal vaginal bleeding to their physicians. Any postmenopausal vaginal bleeding or spotting is suspicious and is evaluated. Currently available diagnostic tests include endometrial biopsy or dilation and curettage with hysteroscopy; however, there are no specific biomarkers available as prognostic indicators for endometrial cancer. The most extensively studied biologic markers in endometrial carcinoma are estrogen and progesterone receptors. It has been shown that high levels of estrogen and progesterone receptors directly correlate with better tumor differentiation, less myometrial invasion, and a lower incidence of nodal metastases and that they independently predict better survival (Zaino, R. J., et al., Gynecol. Oncol., 16, 196-208 (1983); Creasman, W. T., Cancer, 71 (Suppl.):1467-1470 (1993)).

In 1947, Gusberg described the significance of the precursors of endometrial cancer and coined the term “adenomatous hyperplasia” (Gusberg, S. B., Am. J. Obstet. Gynecol., 54, 905-927 (1947)). The frequency of adenomatous hyperplasia as a precursor to well-differentiated endometrial cancer has increased yearly. A very careful histologic examination and regular follow-up visits are prerequisites for this method of treatment, since hyperplasia and cancer can coexist in the same patient.

Therefore, there is an ongoing need for a simple and effective screening method for identifying patients that have tumors that will likely progress to malignancy.

### SUMMARY OF THE INVENTION

The present invention provides a method for diagnosing a predisposition for neoplasia in a patient by contacting a biological sample potentially comprising a complement regulatory protein (CRP) from the patient with a anti-CRP antibody to form an CRP-antibody complex; and measuring the quantity of CRP-antibody complex in the biological sample as compared to a normal control level, wherein the quantity of CRP-antibody complex as compared to a normal control is indicative for a predisposition for neoplasia. The CRP may be cell membrane-associated or secretory. The neoplasia may be malignant. The CRP may be CD35 (complement receptor type 1, CR1), CD46 (membrane cofactor protein, MCP), CD55 (also decay accelerating factor, DAF) or CD59 (membrane attack complex inhibitory factor, MACIF). The anti-CRP antibody may be immobilized on a solid surface. The anti-CRP antibody may be a detectable label or a binding site for a detectable label to form detectable complexes. The detectable label may be an enzyme label, or a fluorogenic compound. The binding site for the detectable label may be biotin, avidin or streptavidin. The biological sample may be a physiological fluid or tissue sample, such as a tissue sample from a breast, cervical, ovarian, prostate or endometrial tumor.

The present invention also provides a method for diagnosing a predisposition for neoplasia in a patient by contacting a biological sample potentially comprising CRP from the patient with a solid surface having immobilized thereon anti-CRP antibodies, so that the CRP binds to the anti-CRP antibodies; contacting labeled CRP, which comprises a detectable label or a binding site for a detectable label, with the solid surface, so that the labeled CRP binds to free antibodies on the solid surface to form detectable complexes; and detecting the complexes, wherein the quantity of the complexes is inversely proportional to the amount of CRP in the biological sample, wherein the quantity of CRP-antibody complex as compared to a normal control is indicative for a predisposition for neoplasia. The neoplasia may be malignant. The CRP may be CD35, CD46, CD55 or CD59. The detectable label may be an enzyme label or a fluorogenic compound. The binding site for the detectable label may be biotin, avidin or streptavidin. The biological sample may be a physiological fluid or tissue sample, such as a tissue sample from a breast, cervical, ovarian, prostate or endometrial tumor.

The present invention also provides an article of manufacture for diagnosing a predisposition for neoplasia in a patient comprising packaging material, and a diagnostic kit and instructions within the packaging material, wherein the diagnostic kit comprises anti-CRP antibody, and a means for measuring the quantity of CRP-antibody complexes in a biological sample from a patient wherein the quantity of CRP-antibody complex as compared to a normal control is indicative for a predisposition for neoplasia, and wherein the instructions that indicate that the diagnostic kit can be used to diagnose a predisposition for neoplasia in a patient. The neoplasia may be malignant. The CRP may be CD35, CD46, CD55 or CD59. The kit may also contain a solid substrate. The anti-CRP antibody of the kit may be immobilized on a solid surface. The anti-CRP antibody may be a detectable label or a binding site for a detectable label to form detectable complexes. The detectable label may be an enzyme label. The detectable label may be a fluorogenic compound. Alternatively, the binding site for the detectable label may be biotin, avidin or streptavidin. The biological sample may be a physiological fluid or tissue sample, such as a tissue sample from a breast, cervical, ovarian, prostate or endometrial tumor.

The present invention further provides a method for early diagnosis of a premalignant lesion in a patient comprising contacting a biological sample potentially comprising a cell membrane-associated complement regulatory protein (CRP) from the patient with a anti-CRP antibody to form an CRP-antibody complex; and measuring the quantity of CRP-antibody complex in the biological fluid as compared to a normal control level, wherein the quantity of CRP-antibody complex as compared to a normal control is indicative for a predisposition for developing a malignant lesion.

Moreover, the present invention provides a method for determining the prognosis of a malignant lesion in a patient comprising contacting a biological sample potentially comprising a cell membrane-associated complement regulatory protein (CRP) from the patient with a anti-CRP antibody to form an CRP-antibody complex; and measuring the quantity of CRP-antibody complex in the biological fluid as compared to a normal control level, wherein the quantity of CRP-antibody complex as compared to a normal control is predictive of the likelihood of success of a particular course of treatment of the malignant lesion.

### BRIEF DESCRIPTION OF THE DRAWINGS

FIG. 1. Complement CR1: Graphic representation of amount of CR1 expression in benign and malignant endometrial tissue samples.

FIG. 2 Complement MCP: Graphic representation of amount of MCP expression in benign and malignant endometrial tissues samples.

FIG. 3 Complement DAF: Graphic representation of amount of DAF expression in benign and malignant endometrial tissue samples.

FIG. 4. Complement MACIF: Graphic representation of amount of MACIF expression in benign and malignant endometrial tissue samples.

FIG. 5. TNF-alpha modulation of DAF (CD55) on MCF-7 breast cancer cells.

### DETAILED DESCRIPTION

One of the physiologic roles of the complement system is the lysis of foreign cells, including tumor cells. This cytolytic process is activated by the interaction of more than 20 plasma proteins in two pathways; the classical and alternative pathways. The classical pathway is triggered by antigen-antibody complexes, while the alternative pathway is triggered by foreign surfaces including tumor cells (Kumar, S., et al., Cancer Res. 53, 348-353 (1993)). Activation of either pathway leads to the formation of a biomolecular complex, designated C3 convertase (Lublin, D. M., et al. Annu. Rev. Immunol., 7, 35-58 (1989)). The final steps in binding of complement proteins to the cell membrane are common to both pathways and consist of the sequential assembly of the C5b, C6, C7, C8 and C9 components into the membrane attack complex (MAC). This pore-forming MAC inserts into the lipid bilayer of the plasma membrane and causes cell lysis (Kumar, S., et al. Baglioun, Cancer Res. 53, 348-353 (1993)).

To protect themselves from autologous complement-mediated damage, normal human tissues express cell membrane-associated complement regulatory proteins (CRPs). CRPs have activity at two levels within the complement cascade, at either the C3 convertase or MAC formation level. CRPs that operate at the C3 convertase level are known as the regulators of complement activation (RCA) group (Koretz, K., et al., Br. J. Cancer, 68, 926-931 (1993)). Complement receptor type 1 (CR1, CD35), membrane cofactor protein (MCP, CD46), and decay-accelerating factor (DAF, CD55) are members of the RCA group of proteins. CR1 is a polymorphic (190-280 kDa) receptor whose primary ligands are C3b and C4b. CR1 has limited tissue distribution, and is found primarily on hematopoietic cells in peripheral blood. Its major function is to bind, process, and transport C3b/C4b-coated immune complexes and particles. MCP is also a widely distributed C3b/C4b-binding dimeric protein with molecular masses of 50-58 kDa (lower form) and 59-68 kDa (upper form), and serves as a cofactor for the plasma serine protease factor I, which irreversibly inactivates C3b and C4b (Bjorge, L., et al., Cancer Immunol. Immunother., 42, 185-192 (1996)). DAF is a 70 kDa glycolipid-anchored membrane-bound CRP with a wide tissue distribution that possesses regulatory activity for the C3 convertases. DAF protects the host tissue by inhibiting assembly (C2a with C4b or Bb with C3b) and/or promoting dissociation of preformed C3 convertases on the same cell (Hourcade, D., et al., Adv. Immunol, 45, 381-416 (1989)). The CRP that operates at the MAC level is known as protectin [membrane attack complex inhibitory factor (MACIF), CD59]. Protectin is an 18- to 20-kDa phosphatidylinositol-anchored glycoprotein in the cell membrane. MACIF inhibits formation of terminal MAC on complement by binding to C8 and C9 molecules and disturbing the C8:C9 ratio in the MAC (Koretz, K., et al., Br. J. Cancer, 68, 926-931 (1993)). These CRPs collectively play a leading role in the immune system both in identification of and in removal of foreign agents, including microorganisms and tumor cells (Bjorge, L., et al., Cancer Immunol. Immunother., 42, 185-192 (1996)).

Decay accelerating factor (DAF, CD55) is a cell-associated complement-regulatory protein that inhibits complement activation and thus protects the autologous tissues from the cytotoxic effects of complement. DAF has been previously associated with paroxysmal nocturnal hemoglobinuria (PNH), as decreased expression of DAF is correlated with presence of the disease. However, PNH is characterized, in part, by lysis of red blood cells (RBCs), and it has been shown that RBCs which do not express DAF (Inab phenotype) often survive complement attack and lysis.

DAF is associated with the Cromer blood group antigens, which are located at various positions along the DAF molecule. It has been characterized as a glycosylphosphatidylinositol (GPI)-anchored membrane protein that inhibits both the classical and alternative pathways of complement activation, its chromosomal location has been identified as band q32.8,9 of human chromosome 1, and its sequence has been reported (Medof, M. E., et al. Proc Natl. Acad. Sci. USA (1987)84: 2007-11). In conjunction with CD59 (protectin), CD46 (membrane cofactor protein), and CD35 (complement receptor type 1 (CR1)), it participates in the regulation of complement activity in the immune response.

Membrane-associated complement regulatory proteins such as CD55 (DAF), CD46 (MCP), CD35 (CR1) and CD59, which show an important mechanism of self protection and render autologous cells insensitive to the action of complement that appears to be overexpressed on certain tumors. In 1988, (Cheung et al., 1988, J. Clin. Invest., 81:1122-1128) showed that one such CRP-decay accelerating factor (DAF; CD55)-expressed on cultured human tumor cell lines protect cells from complement-mediated cytotoxicity. The present inventors have shown that CRP's like CR-1, DAF, MCP and CD59 are expressed in the human genital tract tissues including the endometrium and the cervix and these CRP's in uterine adenocarcinoma samples were found in vivo on breast tumors as well as breast cancer cell lines (Hakulinen et al., 1997, Lab. Invest., 71(6):820-827). Cells expressing low levels of CRP's can be killed in the presence of complement; cells with high level of CRP's are resistant to complement-mediated killing, (Kaul et al., 1995, Infect. Immun, 64(2):611-615).

Hormones and Breast Cancer: The regulation of normal breast development, breast carcinogenesis, and growth and progression of breast cancer seem to depend upon response to hormonal factors. The now classical observations of Beatson in the 1890's (Beatson et al., 1896, Lancet, 2:104-107) established the importance of endocrine influences in the growth control of the breast cancer. Perimenarchal loss of ovarian function can result in a decrease in breast cancer risk by a factor of 100 to about that found in men (J. B. Brown, 1981, In: Branbury Report 8: Hormones and Cancer, Cold Spring Harbor Lab., pp 33-56). Furthermore, ovariectomy and/or anti-estrogenic and antiprogestational drugs have been successfully used in the treatment of breast cancer (Iino et al., 1990, In: Regulatory Mechanisms in Breast Cancer, Edited by Lippman M E and Dickson R B. Kluwer Acad. Pub. Norwell, Miss. pp 221-238). The breast has a tightly regulated pattern of growth that is primarily under the control of steroid hormones. The rate of cell proliferation in postmenopausal breast is considerably less than that in premenopausal breast (Meyer et al., 1982, Cancer, 50:746-751.) Using normal breast epithelial cells during different phases of menstrual cycle, both estrogen and progesterone have been shown to be mitogenic and therefore may contribute to increased breast cancer risks. It is becoming increasingly apparent that hormones play an important and perhaps as yet unappreciated role in the biology of breast cancer. Synthetic progestins have been extensively used in human breast cancer (Pike et al, 1993, Epidemiol. Rev. 15:17-35 and Lober et al., 1981, Acta Obstet Gynecol. Scand (suppl) 101:39-46.), but their mechanism is not well understood. High-dose progestin therapy is receiving a renewed interest for the treatment of advanced breast cancer, either as a first-line or second-line endocrine therapy. This renewed interest is due to low toxicities associated with high-dose progestins and to an efficacy similar to that of tamoxifen (Sedlacek S. Overview of megestral acetate treatment of breast cancer. Sem. Oncol., 15:3-13). Progestins have been observed to be anti-proliferative (Poulin et al., 1989, Breast Cancer Res. Treatment, 13:265-276), behaving similar to antiestrogen tamoxifen by inducing cells to accumulate in the G1 of cell cycle (Sutherland et al., 1988, Cancer Res., 48:5084-5091).

Recent studies indicate that murine mammary gland carcinogenesis is critically dependent on progesterone receptor function (Lydon et al., 1999, Cancer Res., 59(17):4276-4284), as observed in progesterone receptor knockout (PRKO) mouse model (Lydon et al., 1999, Cancer Res., 59(17):4276-4284), thus providing strong support for the role of progesterones in mammary gland tumorogenesis.

Cytokines and Breast Cancer: Multifunctional cytokines play important and only partially defined roles in mammary tumor development and progression. Over the last few years, the role of cytokines in cancer has been the subject of numerous investigations. It has been reported that, in the mammary gland, cytokines play a role in growth and differentiation, extracellular matrix production, angiogenesis and as immunomodulating factors. Recently, it has been shown that epithelial cells of the normal mammary gland produce constitutively IL-6, IL-8 and a non-secreted form of TNF. Primary cultures from ductal carcinomas produce reduced amounts of IL-6 as compared to primary cultures obtained from the normal mammary gland (Suzuki et al, 1999, Cancer Lett., 140:(1-2):161-167.). Mammary epithelial cells constitutively produce not only IL-6, but also IL-8 and a non-secreted form of TNF, further studies are needed to ascertain if the expression of the ER and PR differentiation markers is also associated with altered expression of the above cytokines (Fontanini et al., 1999, Br. J. Cancer, 80(3-4):579-584.).

TNF-α and IFN-γ function as effector molecules released by activated cytotoxic CD8+ and CD4+, lymphocytes and NK cells to reject tumor cells (Lee et al. 1996, J. Immunol., 157(5):1919-1925; Goedegebuure et al., 1997, Cell Immunol., 175(2):150-156; Kaplan et al., 1998, Proc. Natl Acad. Sci. USA, 95(13):7556-7561; Seo et al., 1998, J. Immunol., 161(8):4138-4145; Suzuki et al., 1999, Cancer Lett., 140(1-2): 161-167; and Jager et al., 1999, Int. J. Cancer, 81(6):979-984). TNF-y and IFN-y therefore might be present at various concentrations in tumor environment infiltrated by T lymphocytes and NK cells.

Data suggests that TNF-α and IFN-γ, released by cytotoxic lymphocytes in the course of tumor cell rejection, seems to promote immune escape of tumor cells via induction of FasL expression (Naujokat et al., 1999, Biochem. Biophysical Res. Commun., 264:813-819). TNF-α belongs to a growing family of molecules that have fundamental roles in immune and development networks. These molecules occur in trimeric forms and most of them exist and act as soluble and as transmembrane cell surface proteins. TNF-α is expressed by numerous cell types as a 26-kDa type II transmembrane cell surface proteins. TNF-α is expressed by numerous cell types as a 26-kDa type II transmembrane (mTNF-α) and as a 17-kDa soluble form (sTNF-α) which results from the shedding of mTNF-α by metalloproteinases. In addition to potent immunomodulatory and pro-inflammatory properties, sTNF-α is involved in the lysis of a wide range of normal, infected, or transformed cells. In contrast to the systemic activity of sTNF-α, mTNF-α acts in situations of juxtacrine intercellular signaling by killing tumoral and infected cells in a cell-to-cell contact-dependant manner (Caron et al., 1999, Eur. J. Immunol., 29:3588-3595).

In a recent study, IFN-γ-producing tumor cells had reduced tumorigenicity and were rejected by syngeneic mice. Mechanism of host immune response against two interferon-gamma (IFN-γ) gene-transduced tumors, plasmacytoma MOPC104E (Mu gamma) and mammary cancer SC115 (K gamma), which originally had weak immunogenicity was checked. Specific protection against subsequent challenge with the respective parental tumor cells was demonstrated in mice which rejected the IFN-γ-producing tumor cells. Cultured lymphocytes derived from immunized mouse spleens had cytotoxic T cells activity against parental tumor cells, as well as against cells that produced IFN-γ. These findings indicate that the antitumor effects are mediated by cytotoxic T cells and, partly, by helper T cells, and that locally secreted IFN-γ plays an important role in generating these effector cells (Teramura et al., 1993, Jpn. J. Cancer Res., 84(6):689-696). Vascular endothelium plays an important role in the pathophysiology of tumor metastasis. A recent report has found that both TNF-α and IFN-γ increases the expression of DAF (CD55) on vascular endothelial cells and but not those of other CRP's such as CD 59 or CD 46 (Mason et al., 1999, Blood; September 1; 94(5) 1673-1682).

Multiple lines of evidence implicate steroid hormone and growth factor cross-talk as a modulator of endocrine response in breast cancer and that aberrations in growth factor signaling pathways are a common element in the endocrine resistant phenotype (Nicholson et al., 1999, Endocr. Relat. Cancer, 6(3):373-387). Delineation of these relationships is thus an important diagnostic goal in cancer research, while the targeting of aberrant growth factor signaling holds the promise of improving therapeutic response rates. ER-positive breast cancer cells lines, such as MCF-7, produce growth factors that may act in an autocrine and/or paracrine fashion to influence the proliferation and responsiveness of breast cancer may actually be mediated by hormone-induced growth factors. By binding to specific membrane receptors, growth factors initiate a complex signal transduction cascade that involves sequential phosphorylation-dephosphorylation reactions (Stoica et al., Endocrinol., 138:1498-1505). In the breast, data from numerous laboratories suggest that cross-talk exist between PR and growth factor and cytokine signaling pathways at multiple levels (Lange et al., 1999, Mol. Endocrinol., 13(6):829-836). At the cell surface (level 1), progestins upregulate growth factor and cytokine receptors. Progestins in the cytoplasm (level 2) have been found to regular several intracellular effectors by increasing the levels of Stat5 and control the activity of key genes involved in breast cell fate (Jurianz et al., Mol. Immunol., September-October; 36(13-14):929-939). Thus, apart from hormonal regulation of CRPs, cytokine regulation also needs to be studied during cytokine response to tumor cells.

TNF-α is a macrophage-derived cytokine that causes necrosis of tumors in experimental models. Although, TNF-α is well known for its anti-proliferative action, the mechanisms explaining these phenotropic effects have not been well characterized. Important regulatory proteins such as CRP's and cytokines, whose expression may vary in tissue-specific ways, seem to work in concert with hormones to decide cell fate. It is becoming critically important that the subtleties of the mechanisms of action of hormones be clearly understood in breast tissues.

Immunoassays

The present invention provides a method for assaying the presence or the level of CRP in a biological sample containing CRF to diagnose tumors that will likely progress to malignancy, as certain levels of CRF are predictive of tumor transformation into a malignant tumor. The present invention further provides a method for determining the prognosis of a malignant lesion in a patient, as tumors with certain CRF levels are more responsive to certain types of treatment. Using the methods described in Example 1 below, the threshold values in endometrial tissue samples, based on optical density (OD) as measured by computer based image analysis system, are about: CR1=7.25, CD46=20.0; CD55=19.0 and CD59=22.5. If the OD value is below this threshhold, then the tumor is not likely to become malignant.

Examples of immunoassays that can be employed to determine the relative or absolute amount of CRF in a biological sample include those assay methods, formats and kits disclosed in U.S. Pat. No. 5,516,639.

Examples of immunoassays that can be employed to determine the relative or absolute amount of CRF in a biological sample include those assay methods, formats and kits disclosed in U.S. Pat. No. 5,516,639. CRF analytes may be distinguished from other sample components by reacting the analyte with a specific receptor for that analyte. Assays that utilize specific receptors to distinguish and quantify analytes are often called specific binding assays. The analyte of the present invention may be detected using a variety of specific binding assay formats. For example, various direct-binding assays may be employed. In such assays, receptors, such as antibodies or other binding proteins, are chemically coupled to make a cross-linked protein complex and the complex is immobilized on a solid phase. The immobilized chemically cross-linked protein complexes are contacted with a sample containing the analyte of interest, which may be distinguished from other components found in the sample. For example, an antibody specific for a CRF can be immobilized on the surface of a solid substrate and used as a capture antibody to specifically bind to CRF in a biological fluid. Suitable substrates include particulate substrates such as polystyrene beads, the wells of plastic microtiter plates, paper or synthetic fiber test strips and the like. The immobilized antibody can then be contacted with the test sample to be assayed, e.g., with a biological fluid such as plasma, serum, tears, urine or the like. The resulting antibody-CRF binary complex can then be contacted with an anti-CRF antibody, such as rabbit anti-CRF serum.

Following binding of the analyte by the immobilized complex, the solid phase may be washed and then contacted with an indicator, such as a labeled conjugate. The conjugate comprises an antibody, antibody fragment, binding protein or analyte depending on assay format, and the label is a florescent, enzymated, calorimetric, radiometric or other labeling molecule that is associated either directly or indirectly with the conjugate. The label may be comprised of an enzymatic compound that produces florescence upon contact with a substrate. The extent to which the indicator is present on the solid support can be correlated with the amount of unknown analyte (see, for example, Tijssen, P., Laboratory Techniques in Biochemistry and Molecular Biology, Practice and Theory of Enzyme Immunoassay, pp. 173-219 (Chapter 10) and pp. 329-384 (Chapter 14), Elsevier Science Publishers, Amsterdam, The Netherlands, (1985).

An anti-CRF monoclonal antibody can be itself coupled to a detectable label of a binding site for a detectable label. For example, the antibodies can be labeled radioisotopically, e.g., by ¹²⁵I, or conjugated directly to a detector enzyme, e.g., alkaline phosphatase or horse radish peroxidase, or can be labeled indirectly with a binding site for a detectable label, e.g., via biotinylation. The biotinylated antibody can then be detected by its ability to bind to a an avidin-linked enzyme. If the second antibody is biotinylated, a detector enzyme conjugated to avidin will be subsequently added. The final step for detecting enzymes conjugated to monoclonal antibody or to avidin its the addition of a substrate appropriate for the enzyme to allow quantitative calorimetric detection of reaction product. The value (read in optical density units) can be converted to fmol of CRF by reference to a standard curve generated in a control assay in which a standard extract of detergent-solubilized CRF is added in graded concentrations to the immobilized anti-CRF monoclonal antibody.

The present invention may use many other assay formats, such as competitive immunoassays, bead agglomeration assays and sandwich-type immunoassays, such as ELISA, as would be recognized by the art.

In competitive assay formats, the solid phase containing immobilized chemically cross-linked protein complexes with specificity for a selected analyte is contacted with a sample presumably containing such analyte and with a specific competitive reagent. The specific competitive reagent may be a labeled analog of the analyte. In this specific embodiment, the labeled analog competes with the sample analyte for binding to a receptor immobilized on the solid phase.

In the alternative, an analyte may be coupled to a solid phase and contacted with a sample and with a specific competitive cross-linked protein reagent, for example, a labeled receptor for the analyte. In this format, sample analyte competes with solid phase analyte for binding with soluble labeled cross-linked receptor. In both embodiments, the amount of label bound to the solid phase after washing provides an indication of the levels of analyte in the sample. That is, the amount of analyte in a sample is inversely proportional to the amount of analyte in the sample.

Another embodiment of the present invention is a diagnostic kit for detecting or determining the presence of CRF in a biological sample. Immobilized antibodies and labeled antibodies are conveniently packaged in kit form, wherein two or more of the various immunoreagents will be separately packaged in preselected amounts, within the outer packaging of the kit, which may be a box, envelope, or the like. The packaging also preferably comprises instruction means, such as a printed insert, a label, a tag, a cassette tape and the like instructing the user in the practice of the assay format. For example, one such diagnostic kit for detecting or determining the presence of CRF comprises packaging containing, separately packaged: (a) a solid surface, such as a fibrous test strip, a multi-well microliter plate, a test tube, or beads, having bound thereto antibodies to CRF; and (b) a known amount of antibodies specific to CRF, wherein said antibodies comprise a detectable label, or a binding site for a detectable label.

Solid Supports

A solid support useful in the present invention is a matrix of material in a substantially fixed arrangement. Exemplary solid supports include glasses, plastics, polymers, metals, metalloids, ceramics, organics, etc. Solid supports can be flat or planar, or can have substantially different conformations. For example, the substrate can exist as particles, beads, strands, precipitates, gels, sheets, tubing, spheres, containers, capillaries, pads, slices, films, plates, slides, etc. Magnetic beads or particles, such as magnetic latex beads and iron oxide particles, are examples of solid substrates that can be used in the methods of the invention. Magnetic particles are described in, for example, U.S. Pat. No. 4,672,040, and are commercially available from, for example, PerSeptive Biosystems, Inc. (Framingham Mass.), Ciba Corning (Medfield Mass.), Bangs Laboratories (Carmel Ind.), and BioQuest, Inc. (Atkinson N.H.).

Indicator Labels

The labels used in the assays of invention can be primary labels (where the label comprises an element which is detected directly) or secondary labels (where the detected label binds to a primary label, e.g., as is common in immunological labeling). An introduction to labels, labeling procedures and detection of labels is found in Polak and Van Noorden (1997) Introduction to Immunocytochemistry, second edition, Springer Verlag, N.Y. and in Haugland (1996) Handbook of Fluorescent Probes and Research Chemicals, a combined handbook and catalogue Published by Molecular Probes, Inc., Eugene, Oreg. Primary and secondary labels can include undetected elements as well as detected elements. Useful primary and secondary labels in the present invention can include spectral labels such as fluorescent dyes (e.g., fluorescein and derivatives such as fluorescein isothiocyanate (FITC) and Oregon Green™, rhodamine and derivatives (e.g., Texas red, tetramethylrhodamine isothiocyanate (TRITC), etc.), digoxigenin, biotin, phycoerythrin, AMCA, CyDyes™, and the like), radiolabels (e.g., ³H, ¹²⁵I, ³⁵S, ¹⁴C, ³²P, ³³P), enzymes (e.g., horse-radish peroxidase, alkaline phosphatase) spectral colorimetric labels such as colloidal gold or colored glass or plastic (e.g. polystyrene, polypropylene, latex) beads. The label may be coupled directly or indirectly to a component of the detection assay (e.g., the labeling nucleic acid) according to methods well known in the art. As indicated above, a wide variety of labels may be used, with the choice of label depending on sensitivity required, ease of conjugation with the compound, stability requirements, available instrumentation, and disposal provisions. In general, a detector which monitors an analyte-receptor complex is adapted to the particular label which is used. Typical detectors include spectrophotometers, phototubes and photodiodes, microscopes, scintillation counters, cameras, film and the like, as well as combinations thereof. Examples of suitable detectors are widely available from a variety of commercial sources known to persons of skill. Commonly, an optical image of a substrate comprising bound analyte is digitized for subsequent computer analysis.

Preferred labels include those which utilize 1) chemiluminescence (using Horseradish Peroxidase and/or Alkaline Phosphatase with substrates that produce photons as breakdown products) with kits being available, e.g., from Molecular Probes, Amersham, Boehringer-Mannheim, and Life Technologies/Gibco BRL; 2) color production (using both Horseradish Peroxidase and/or Alkaline Phosphatase with substrates that produce a colored precipitate) (kits available from Life Technologies/Gibco BRL, and Boehringer-Mannheim); 3) hemifluorescence using, e.g., Alkaline Phosphatase and the substrate AttoPhos (Amersham) or other substrates that produce fluorescent products, 4) Fluorescence (e.g., using Cy-5 (Amersham), fluorescein, and other fluorescent tags); 5) radioactivity using kinase enzymes or other approaches. Other methods for labeling and detection will be readily apparent to one skilled in the art.

Fluorescent labels are highly preferred labels, having the advantage of requiring fewer precautions in handling, and being amendable to high-throughput visualization techniques (optical analysis including digitization of the image for analysis in an integrated system comprising a computer). Preferred labels are typically characterized by one or more of the following: high sensitivity, high stability, low background, low environmental sensitivity and high specificity in labeling. Fluorescent moieties, which are incorporated into the labels of the invention, are generally are known, including Texas red, dixogenin, biotin, 1- and 2-aminonaphthalene, p,p′-diaminostilbenes, pyrenes, quaternary phenanthridine salts, 9-aminoacridines, p,p′-diaminobenzophenone imines, anthracenes, oxacarbocyanine, merocyanine, 3-aminoequilenin, perylene, bis-benzoxazole, bis-p-oxazolyl benzene, 1,2-benzophenazin, retinol, bis-3-aminopyridinium salts, hellebrigenin, tetracycline, sterophenol, benzimidazolylphenylamine, 2-oxo-3-chromen, indole, xanthen, 7-hydroxycoumarin, phenoxazine, calicylate, strophanthidin, porphyrins, triarylmethanes, flavin and many others. Many fluorescent tags are commercially available from the SIGMA Chemical Company (Saint Louis, Mo.), Molecular Probes, R&D systems (Minneapolis, Minn.), Pharmacia LKB Biotechnology (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersberg, Md.), Fluka ChemicaBiochemika Analytika (Fluka Chemie AG, Buchs, Switzerland), and Applied Biosystems (Foster City, Calif.), as well as many other commercial sources known to one of skill.

Most typically, the analyte is measured by quantifying the amount of label fixed to the solid support by the capture of the linked complex between analyte and receptor. Typically, the presence in the reaction mixture of an analyte-receptor complex will increase or decrease the amount of label fixed to the solid support relative to a control reaction which does not comprise the analyte. Means of detecting and quantifying labels are well known to those of skill in the art. Thus, for example, where the label is a radioactive label, means for detection include a scintillation counter or photographic film as in autoradiography. Where the label is optically detectable, typical detectors include microscopes, cameras, phototubes and photodiodes and many other detection systems which are widely available.

Biological Samples

Biological samples that can be used in the present invention include physiological fluids or tissue samples. Physiological fluids from patients include plasma, serum, tears, urine, and the like. A tissue sample may be obtained such as by biopsy.

The following examples are intended to illustrate but not limit the invention.

### EXAMPLE 1

### Expression of CRPs in Endometrial Tissue

Materials and Methods

Tissue Procurement and Preparation

Endometrial tissue samples were collected from 54 patients between October 1994 and January 1997 after obtaining proper consent. Thirty-one of the fifty-four patients had final diagnosis of benign endometrium, and provided the basis for the control group. Twenty-three of the fifty-four patients had biopsy-proven diagnosis of adenocarcinoma of the endometrium, and underwent complete surgical staging. Surgical staging included pelvic washings, exploration of the upper abdomen, total abdominal hysterectomy, bilateral salpingo-oopherectomy, and sampling of pelvic and paraaortic lymph nodes. This group of patients constituted our experimental group. The collected endometrial tissue was frozen at −70° C. Serial cryosections of 5 μm were obtained from the collected tissue samples and quick fixed in ice-cold acetone and stored at −20° C. for immunohistochemical (IHC) staining at a later date. Hemotoxylin and eosin-stained parallel sections from each patient were reviewed by two pathologists to confirm histologic diagnosis.

Antibodies and Reagents

Mouse monoclonal antibodies for human CR1 (CD 35), MACIF (CD 59), and DAF (CD 55) were purchased commercially. Anti-CR1 (clone E11) and anti-MACIF [clone p282 (H19)] were obtained from Pharmingen (San Diego, Calif.), and used at a dilution of 1:100 for a concentration of 1 μg/ml. Anti-DAF (clone 1C6) was purchased from WAKO BioProducts (Richmond, Va.) and diluted to 1:1000, for a final concentration of 1 μg/ml. MCP (CD 46), mouse monoclonal antibody was generously provided by Dr. John P. Atkinson at Washington University School of Medicine (St. Louis, Mo.). The final dilution used in IHC staining of anti-MCP was 1:1000, for a concentration of 1 μg/ml.

Immunohistochemical Staining

Frozen sections of endometrial tissues were stained with antibodies to CR1, MCP, DAF, and MACIF using the avidin-biotin-peroxidase complex (ABC) as described before (Kaul, A. et al., Am. J. Reprod. Immunol., 34, 236-240 (1995)). The slides were removed from the −20° C. freezer and brought to room temperature over 5 min. Before staining, the tissue sections were fixed in ice-cold acetone for 10 min. After a rinse in PBS, the tissue samples were blocked at room temperature with normal goat serum (50 μl/slide) for 20-25 min. After draining off the normal goat serum, primary antibodies and control buffers were placed onto the tissue sections (50 μl). The slides were then incubated for 1 h in a humid chamber. The slides were washed three times with PBS+Tween 20 buffer. Next, the secondary antibody, goat anti-mouse IgG, was applied, and further incubation was carried out in a humid chamber for 15 min. After another washing step with PbS+Tween 20, streptavidin was applied to each tissue section and incubated in the humid chamber for another 15 min. At the end of this incubation, the washing step was repeated with PBS+Tween 20 as above, the slides were allowed to air-dry, and excess fluid was wiped clean. Next, diaminobenzidine (DAB) was placed on the slides to highlight areas of receptor presence. Slides were washed with PBS, counterstained with Mayer's hemotoxylin (Sigma-Aldrich Chemical Co., St. Louis, Mo.), and mounted in a xylene-based mounting medium (Permount), and coverslipped. Control sections included all the above steps except the primary antibody was replaced by an appropriate buffer.

Image Analysis

Stained tissue sections were quantitated for CRP content using a microcomputer-based image analysis system as described earlier (Kaul, A. et al., Am. J. Reprod. Immunol., 34, 236-240 (1995)). The Samba 4000 system (Imaging Products International, San Francisco, Calif.) was used to measure the stained endometrial tissue sections for integrated optical density (OD). A total of five fields at 20× magnification were measured in stained and unstained serial sections. The average OD per each stained and unstained section was computed. The difference in OD between antibody-stained and unstained parallel sections was used as a measure of the protein content in the benign and malignant tissue samples.

Statistical Analysis

Comparison was made for each protein studied between the benign and malignant endometrial samples. An unpaired Student t test was used to analyze the data and determine statistical significance. Statistical computations were performed and graphics developed using JMP software (SAS Institute, Cary, N.C.).

Although a total of 54 specimens were collected, only 48 samples were included in this study. Six patient specimens were eliminated from our study. Exclusion from the study resulted secondary to insufficient amount of endometrial tissue (3 benign cases), cryosections that were too bloody and would falsely elevate OD measurement for protein content (1 benign and 1 malignant case), and in one specimen designated malignant, no evidence of carcinoma present. Therefore, the evaluable patients included 27 with benign diagnosis and 21 with malignant endometrial tissue.

The 27 benign endometrial tissue samples provided our control group for the study. The mean age was 51 years (range 34-80). The distribution of histologic diagnoses was 14 proliferative, 4 secretory, and 8 atrophic endometrial samples. Five of twenty-seven patients were on hormone replacement therapy. The following were the clinicopathologic characteristics of the 48 evaluable patients in this study.

Twenty-one patients, whose mean age was 65 (range, 43-83), had malignant endometrial specimens. Histologic evidence of endometriod adenocarcinoma was present in 18 tissue samples, and three samples were consistent with papillary serous carcinoma. When the 21 patients in the malignant sample population were distributed by stage, 12 patients had Stage I disease (2 IA, 8 IB, 2 IC), 3 Stage II (all IIA), 4 Stage III (1 IIIA and 3 IIIC), and 2 Stage IV (1 IVA, 1 IVB). With respect to patient distribution by tumor grade, 10 specimens were G1, 7 G2, and 4 G3. Only 2 of 21 patients with a final diagnosis of malignancy were on hormone therapy at the time of their surgery. There was no significant difference observed when comparing age, histologic diagnosis, stage, grade, depth of myometrial invasion, or hormone therapy status with respect to CRP expression in each of the four proteins studied. Our small sample sizes in these subgroups attributed to this finding. The staining distribution for each of the CRPs was evaluated. The positive controls stained appropriately and the negative controls showed no staining. For all four of the CRPs studied, the IHC staining for each protein was noted to be in a circumferential membranous distribution.

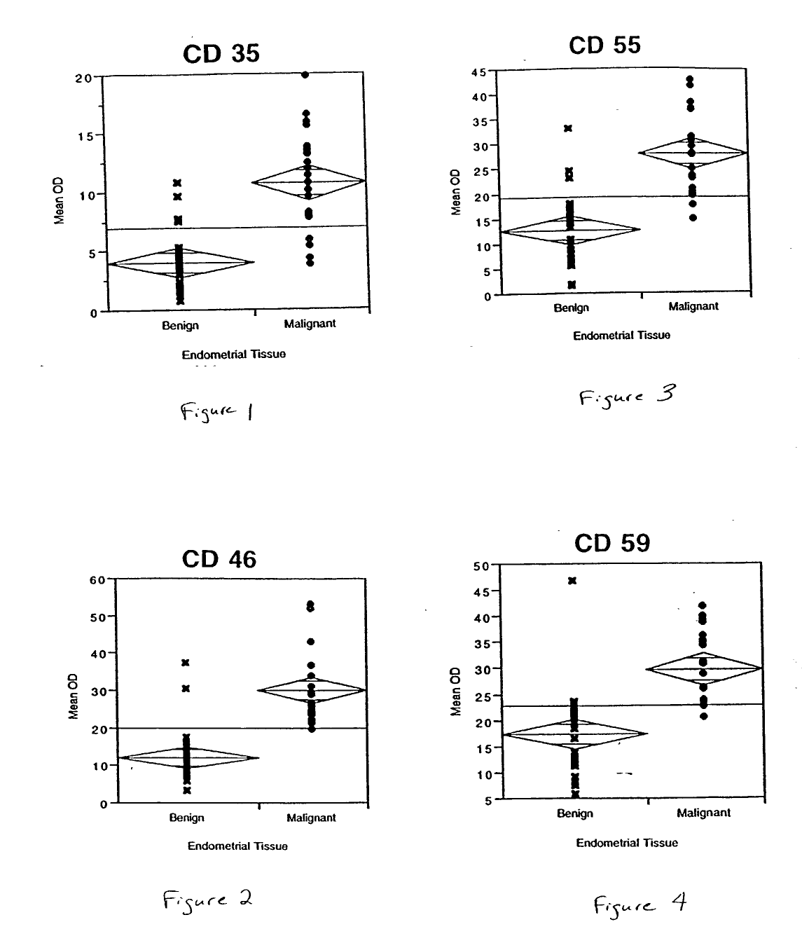

For complement CR1, the benign endometrial samples had a mean OD of 4.04±2.51, and the mean OD for the carcinoma specimens was 10.80±4.27, reaching statistical significance (P<0.0001). A graphic representation of the amount of protein expression observed in the tissue is shown in FIG. 1.

For complement MCP, the benign endometrial samples had a mean OD of 12.24±7.22, and the mean OD for the carcinoma specimens was 30.11±9.21, reaching statistical significant (P>0.0001). A graphic representation of the amount of protein expression observed in the tissue is shown in FIG. 2.

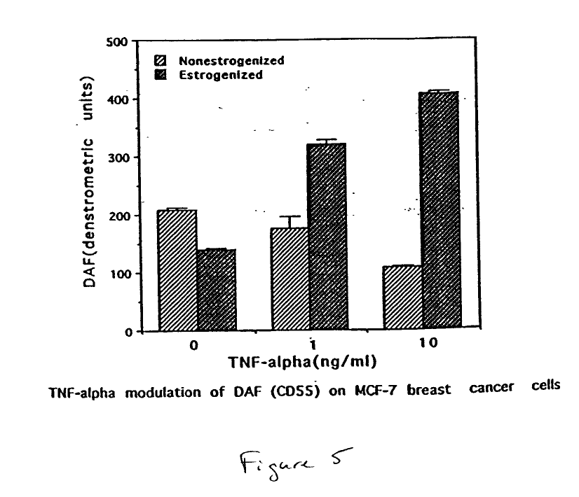

For complement DAF, the benign endometrial specimens had a mean OD of 12.76±6.93, and the mean OD for the carcinoma specimens was 27.96±7.98, reaching statistical significance (P<0.0001). A graphic representation of the amount of protein expression observed in the tissue is shown in FIG. 3.

For complement MACIF, the benign endometrial specimens had a mean OD of 17.56±7.89, and the mean OD for the carcinoma specimens was 29.84±6.93, reaching statistical significance (P<0.0001). A graphic representation of the amount of protein expression observed in the tissue is shown in FIG. 4.

Discussion

The results of the present study demonstrate that human endometrial tissues express membrane-associated complement inhibitors, with the level of expression varying between benign and malignant tissue types. For all four of the CRPs studied (CR1, MCP, DAF, MACIF), there was a statistically significant difference in quantitative protein expression between benign and malignant endometrial samples (P<0.0001). The quantitative value for each CRP in the malignant endometrial tissues reveals significantly higher levels of MCP, DAF, and MACIF in comparison to CR1. This is secondary to the wide tissue distribution of the former three CRPs as compared with the latter, which is found primarily on hematopoietic cells in peripheral blood.

Each of the CRPs inhibits the complement activation cascade at a different point in the scheme of the complement cascade. CR1, DAF, and MCP inhibit the complement system at the C3 convertase level, while protectin inhibits the formation of MAC, thereby inhibiting cell lysis. The proposed mechanism of the CRP is as follows: high levels of protein expression on tumor cells protect these cells from complement-mediated cytotoxicity. In addition to direct tumoricidal actions, complement activation can impact indirectly on tumor growth through effects on vessel permeability, cell trafficking, and possibly sensitization of tumors to cellular effects. Local formation of C3a can increase both blood flow and diffusion of proteins into tumor-containing tissues. The generation of C5a may also increase the influx of phagocytes to tumor sites. The deposition of iC3b (or C3b) on target cell surfaces has been demonstrated to promote cytotoxic activity of lymphocytes (Perlmann, H. et al., J. Exp. Med., 153, 1592-1603 (1981)). The increase in recruitment of effector cells to tumor sites and the enhancement in the efficiency of cell-dependent modalities of tumor killing should contribute to cytotoxicity. Little is presently known about the production of complement inhibitors by tumor cells as a defense against cytotoxic lymphocytes. The finding of the present study suggests a role for these CRPs in tumor resistance to complement-mediated cytolysis.

### EXAMPLE 2

### TNF-α Modulation of DAF on MCF-7 Breast Cancer Cells

Estrogen plays an integral role in the growth of most estrogen receptor positive mammary carcinomas. The proliferative effects of steroids in normal and neoplastic tissues seen in breast cancer may actually be mediated by hormone-induced growth factors. Aberration in growth factors signaling pathways are a common element in the endocrine resistant phenotype and thus affect therapeutic response rates. TNF can alter estrogen-regulated metabolic processes in breast cancer cells leading to growth inhibition.

Estrogen primed and unprimed MCF-7 cells were treated with different concentrations of recombinant TNF-α for 18 hours. Estrogen primed cells were treated overnight with 20 ng/ml β estradiol. DAF protein was extracted by lysis treatment of cells and quantitated, run on SDS-PAGE followed by western blot analysis using anti DAF antibody. Bands were visualized by ECL method (Amersham). DAF levels were quantitated densitometrically and expressed as densitometric units. In MCF-7 cells growth under nonestrogenized conditions, TNF-α downregulated expression of CD55 in a dose dependent manner.

Whereas cells grown under estrogenized conditions, TNF-α upregulated CD55 in a dose dependent manner (FIG. 5). The upregulation of DAF (CD55) under estrogenized conditions and the downregulation under nonestrogenized conditions by TNF-α indicates important interaction between immune and endocrine system—perhaps modulated by estrogen and progesterone receptor expression (Jurianz et al., Mol. Immunol., September-October; 36(13-14):929-939).

### EXAMPLE 3

### Analysis and Quantification of the Levels of DAF in Normal and Neoplastic Human Breast Tissue

The regulation of normal breast development, breast carcinogenesis and growth progression of breast cancer seem to depend upon response to hormonal factors. Elevated levels of DAF are seem in endometrial cancers and is under hormonal control in the human endometrium (see example 1).

Possible differences in density of DAF are correlated with clinical stage of premalignant and malignant lesions. Breast tissue sample are taken from each patient enrolled for this study and analyzed for the expression of DAF.

About 60 normal and 60 neoplastic breast tissue samples are collected. Complete patient history is evaluated including the hormonal status, diet, menopause status, and if menstruating, the timing of the menstrual cycle for an correlation with the laboratory findings. Breast tissues are collected after each prospective patient is approached by an attending physician. The procedures, risks, and long-range benefits of the tissue donation and the project are described to the patient. If for any reason a patient is hesitant to participate, she is excluded. Collected tissue is immediately placed into a plastic container on wet crushed ice and transported at +4° C. to surgical histopathology laboratory. Upon arrival (no longer than 20 minutes) tissue samples are frozen in liquid nitrogen and placed in a −70° C. freezer. Tissue sections (5 μm) are prepared and examined histopathologically. The tissues are fixed in acetone, and used for immunohistochemical staining.

### EXAMPLE 4

### Evaluation of the Levels of Estrogen/Progesterone Receptor with the Levels of DAF and Cytokines in the Human Breast Tissue

Rationale: CRP's such as DAF are under the control of hormonal influence in the human endometrium (Kaul et al., 1995, Infect Immun., 64(2):611-615). Malignant and benign breast tissues are also studied to determine the role of CRP's such as DAF. The expression of DAF is correlated with that of estrogen and progesterone receptors in the collected tissue samples.

The estrogen and progesterone receptors are currently used as an important diagnostic tool in the prognosis of breast cancers. The ER/PR in the collected breast tissue samples are quantitated using immunohistochemical staining procedures. The antibodies for ER and PR are obtained from Dako Corp., Carpentaria, Calif. The value of ER/PR is then correlated with the values obtained by the immunohistochemical staining of parallel tissue section using specific antibodies to identify DAF in these tissues. This correlation is an important factor in comparing DAF levels with either ER or PR or both.

### EXAMPLE 5

### Immunohistochemical Staining Procedures

ABC technique will be employed to stain breast tissue sections for the presence of DAF, TNF-α, IFN-γ, and ER/PR using specific antibody obtained commercially. Section of fixed tissue is carried through the following steps at room temperature. Steps 1, 3, 4, and 5 are followed by three careful washes of three minutes each in Tris buffer, pH 7.4. 1) 10 minutes ice-cold acetone fixation; 2) Normal goat serum diluted 1/10 in Tris buffer for 20 minutes; removal of excess serum; 3) Incubation at room temperature for 1 hour with the specific antibody and isotype control for controls; 4) Incubation at room temperature for 1 hour with biotinylated second antibody (Vector Laboratories, Burlingame, Calif.); 5) Avidin-biotin complex (Vector Lab) for 30 minutes; 6) 0.005% diaminobenzidine tetrahydrochloride (Sigma Chemical Corp., St. Louis, Mo.) in Tris buffer, pH 7.4 (activated by one drop of 30% H₂O₂) for 8 minutes; 7) Counterstain with Meyer's hematoxylin; 8) Mounting and viewing.

Control Procedures: Specific antibody is replaced with isotype control of specific antibody on control parallel sections.

### EXAMPLE 6

### Quantification of DAF and Cytokine Content

Although use of image analysis has been well established in cancer studies, this method has not been used in the field of quantitating DAF. Quantitation of DAF content is done by microcomputer-based image-analysis system. Frozen sections of breast tissues are stained with specific antibody (positive control) and isotype control of the same antibody (as the negative control), by the avidin-biotin peroxidase complex (ABC) technique. Stained sections are measured or integrated optical density (OD). The difference in OD between antibody-stained and isotype control stained parallel sections will be used as a measure of the DAF content. Slides will be examined at various magnifications ranging from 4× to 25× (Murray et al., 1999, Gynecol. Oncol., 76 (In Press)).

Differences between group means are calculated using multivariate analysis (Hotelling T²), and the comparisons are performed using Welch's test or the Student's t-test. Simple linear regression analysis is used to show the degree of linear association between the different cytokines and hormones studied, and a two-tailed probability level of 0.5 is considered significant. Stepwise multiple regression is used to predict cytokine levels in the breast cancer tissue. In stepwise multiple regression analysis a variable is included if its partial regression coefficient is significant at the 0.5 level, and is eliminated if its partial regression coefficient is not significant at 0.10 level.

### EXAMPLE 7

### DAF as a Prognostic Marker in Ductal Carcinoma in situ (DCIS)

DAF expression in breast cells progressively leads through various stages to premalignant, malignant and more advanced stages, which allows the cancer cells to more effectively resist complement mediated killing. DAF can be used as an important immunohistochemical marker to help predict the potential stromal invasion. Over-expression of DAF by breast cancer cell can be used as an early prognostic tool in patients diagnosed with DCIS of the breast.

DAF levels on breast tissue samples form patients who have been diagnosed with DCIS had have had multiple breast tissue samples (at least 3) taken over a period of time are analyzed and quatitated. Tissue sections from at least 25 patients previously diagnosed with DCIS are obtained on glass slides from paraffin embedded breast tissue blocks. Immunohistochemical assay for DAF protein in human breast carcinoma, along with tissue from normal breast and benign diseases are performed. Breast tissues are stained with specific antibody (positive control) and isotype control of the same antibody (as the negative control), by the avidin-biotin peroxidase complex techniques. Stained section are measured for integrated optical density (OD). Although use of image analysis has been well established in cancer studies, this method has not been used in the field of quantitating DAF. Murray K, et al. (2000) Gynecol Oncol. 76. Quantitation of DAF content is done by microcomputer-based image-analysis system. The difference in OD between antibody-stained and isotype control stained parallel sections are used as a measure of the DAF content. Slides are examined at various magnifications ranging from 4× to 25×. The level of DAF on each of the tissue sections is quatitated and correlated with the clinical history of the patient at the time when the tissue was collected and also the histopathology of the breast tissue. Data is reviewed regarding the role of DAF expression as a prognostic variable, and as a predicative factor for response to chemotherapy and/or hormonal therapy.

All publications, patents and patent documents are incorporated by reference herein, as though individually incorporated by reference. The invention has been described with reference to various specific and preferred embodiments and techniques. However, it should be understood that many variations and modifications may be made while remaining within the scope of the invention.

What is claimed is:

1. A method for diagnosing a predisposition for a neoplasia in a patient comprising:
(a) contacting a biological sample potentially comprising a complement regulatory protein (CRP) from the patient with a anti-CRP antibody to form an CRP-antibody complex; and
(b) measuring the quantity of CRP-antibody complex in the biological fluid as compared to a normal control level, wherein the quantity of CRP-antibody complex as compared to a normal control is indicative for a predisposition for developing a neoplasia.

2. The method of claim 1, wherein the neoplasia is malignant.

3. The method of claim 1, wherein the CRP is CD35, CD46, CD55 or CD59.

4. The method of claim 1, wherein the anti-CRP antibody is immobilized on a solid surface.

5. The method of claim 1, wherein the anti-CRP antibody comprises a detectable label or a binding site for a detectable label to form detectable complexes.

6. The method of claim 5 wherein the detectable label is an enzyme label.

7. The method of claim 6 wherein the detectable label is a fluorogenic compound.

8. The method of claim 5 wherein the binding site for the detectable label is biotin, avidin or streptavidin.

9 The method of claim 1 wherein the biological sample is a physiological fluid or a tissue sample.

10. The method of claim 9 wherein the tissue sample is from a breast, cervical, ovarian, prostate or endometrial tumor.

11. A method for diagnosing a predisposition for neoplasia in a patient comprising:
(a) contacting a biological sample potentially comprising a CRP from the patient with a solid surface having immobilized thereon anti-CRP antibodies, so that the CRP binds to the anti-CRP antibodies;
(b) contacting labeled CRP, which comprises a detectable label or a binding site for a detectable label, with the solid surface, so that the labeled DAF binds to free antibodies on the solid surface to form detectable complexes; and
(c) detecting the complexes,
wherein the quantity of the complexes is inversely proportional to the amount of CRP in the biological sample, and wherein the quantity of CRP-antibody complex as compared to a normal control is indicative for a predisposition for neoplasia.

12. The method of claim 11, wherein the neoplasia is malignant.

13. The method of claim 11, wherein the CRP is CD35, CD46, CD55 or CD59.

14. The method of claim 11 wherein the detectable label is an enzyme label.

15. The method of claim 11 wherein the detectable label is a fluorogenic compound.

16. The method of claim 11 wherein the binding site for the detectable label is biotin, avidin or streptavidin.

17. The method of claim 11 wherein the biological sample is a physiological fluid or a tissue sample.

18. The method of claim 17 wherein the tissue sample is from a breast, cervical, ovarian, prostate or endometrial tumor.

19. An article of manufacture for diagnosing a predisposition for neoplasia in a patient having a tumor comprising packaging material, and a diagnostic kit and instructions within the packaging material, wherein the diagnostic kit comprises anti-CRP antibody, and a means for measuring the quantity of CRP-antibody complexes in a biological sample from a patient wherein the quantity of CRP-antibody complex as compared to a normal control is indicative for a predisposition for neoplasia, and wherein the instructions that indicate that the diagnostic kit can be used to diagnose a predisposition for neoplasia in a patient.

20. The article of manufacture of claim 19, wherein the neoplasia is malignant.

21. The article of manufacture of claim 19, further comprising a solid substrate.

22. The article of manufacture of claim 19, wherein the CRP is CD35, CD46, CD55 or CD59.

23. The article of manufacture of claim 19, wherein the anti-CRP antibody is immobilized on a solid surface.

24. The article of manufacture of claim 19, wherein the anti-CRP antibody comprises a detectable label or a binding site for a detectable label to form detectable complexes.

25. The article of manufacture of claim 24, wherein the detectable label is an enzyme label.

26. The article of manufacture of claim 25, wherein the detectable label is a fluorogenic compound.

27. The article of manufacture of claim 26, wherein the binding site for the detectable label is biotin, avidin or streptavidin.

28. The method of claim 19 wherein the biological sample is a physiological fluid or a tissue sample.

29. The method of claim 28 wherein the tissue sample is from a breast, cervical, ovarian, prostate or endometrial tumor.

30. A method for early diagnosis of a premalignant lesion in a patient comprising:
(a) contacting a biological sample potentially comprising a cell membrane-associated complement regulatory protein (CRP) from the patient with a anti-CRP antibody to form an CRP-antibody complex; and
(b) measuring the quantity of CRP-antibody complex in the biological fluid as compared to a normal control level,
wherein the quantity of CRP-antibody complex as compared to a normal control is indicative for a predisposition for developing a malignant lesion.

31. A method for determining the prognosis of a malignant lesion in a patient comprising:
(a) contacting a biological sample potentially comprising a cell membrane-associated complement regulatory protein (CRP) from the patient with a anti-CRP antibody to form an CRP-antibody complex; and
(b) measuring the quantity of CRP-antibody complex in the biological fluid as compared to a normal control level,
wherein the quantity of CRP-antibody complex as compared to a normal control is predictive of the likelihood of success of a particular course of treatment of the malignant lesion.
